(19) United States
(12) Patent Application Publication
(10) Pub. No.: US 2008/0077132 A1
(43) Pub. Date: Mar. 27, 2008

(54) BONE FIXATION DEVICE HAVING INTEGRAL FIXATION MEMBER

(76) Inventor: Robert J. Medoff, Kailau, HI (US)

Correspondence Address: PATZIK, FRANK & SAMOTNY LTD., 150 SOUTH WACKER DRIVE, SUITE 1500, CHICAGO, IL, 60606, US

(21) Appl. No.: 11/534,793

(22) Filed: Sep. 25, 2006

(51) Int. Cl. A61B 17/58

(52) U.S. Cl. 606/60; 606/72; 606/73

(57) ABSTRACT

A bone fixation system consisting of a wire element, such as a be bendable pin, bar or tab, that is bonded to a polymer fixation member over part of its length and extends from the polymer fixation member can be easily secured to a bone or bone fragment with one or more fasteners, such as bone screws, and the wire element buttresses or penetrates a second bone fragment or portion.

### FIELD OF THE INVENTION

The invention relates to a bone fixation system in which a wire element, such as a pin or bar is used for stabilization of one bone section and is bonded as a single composite structure to a fixation member used to fix the wire element to a second bone section. The invention further relates to a bone fixation system in which a wire element is used for stabilization of a first bone section and is bonded as a single composite structure to a fixed post fixation member that fixes the wire element to a second bone section and provides a fixed angle support post to extend from the fixation member into the first bone section.

### BACKGROUND OF THE INVENTION

Existing patents describe and show bendable wires or bars of metal that allow stabilization of a bone fragment. U.S. Pat. No. 5,709,682 shows and describes a type of wire form in which one end of the wire is used to buttress and unstable bone fragment and is held against a stable bone fragment by a screw and washer that compress the wire or bar to the bone. Similarly, U.S. Pat. Nos. 5,941,878, 7,037,308 and 6,113,603 as well as U.S. Publication No. 20050010228 show a U-shaped wire form is described which is secured to a stable bone fragment with a washer. These implants are shown in FIGS. 1-11.

Referring first to FIG. 1, a small fragment clamp is shown. This implant is made of a pair of U-shaped wires 10, 12 with an outer component that sits on the exterior surface of an unstable fragment 14, and an inner wire that projects and supports the inner surface of the unstable fragment 14. The effect of the construct is to grab the fragment 14 like pinching an object between the fingers of a hand. The implant is fixed to an adjacent, stable fragment 16 with a washer 18 and screw 20. The form of the washer 18 is shown in detail in FIG. 4. The effect of the washer 18 is to compress the pair of wire forms 10, 12 against the exterior surface of the stable fragment 16 in order to fix the assembly into place.

FIG. 2 is a similar implant that differs only in that the inner component not only supports the inner surface of the unstable fragment 14, but also provides support of a free articular fragment on its subchondral surface with a pair of legs the project at an angle around 90 degrees to the main plane of the wire form. It also is secured to the stable fragment 16 with a bone screw 20 and washer 18 such as shown in FIG. 4.

FIG. 3 is a volar buttress pin and FIG. 6 is a dorsal buttress pin. These implants are a single U-shaped wire 10 with two legs that project at an angle around 60-100 degrees to the plane of the implant. The function of these implants is to allow the two legs to be impacted into an unstable fragment 14 like a nail or the legs of a table. On the other end, the base of the implant is fixed to the stable bone fragment 16 with a washer 18 and screw 20 as described previously.

FIG. 5 is a post washer 18 that can be used with any of the previous implants and secures the wire form to the proximal or stable fragment 16 in a manner that has been described previously. In addition, this washer has one or more secondary holes 22, 24 that allow a threaded or unthreaded post with a threaded head to be locked into the hole and provide additional stabilization of the unstable fragment 14. This secondary fixation is afforded directly by the fixation post that is locked into the wire and supplements the fixation obtained with the legs of the wire implant.

FIG. 8 is another form of washer 18 used to secure a wire form, in this case a medial mallcolar sled. This washer 18 differs slightly in design from the previous washers in that it captures the wire by two grooves 26, 28 that run the length of the undersurface of the washer 18. Despite this difference, this washer still functions mechanically to compress the wire against the hone (not shown) and gain a frictional lock. In addition, it requires a surgical technique in which a tree washer 18 is placed over an implanted wire form and then the construct held while the bone screw 20 is inserted.

FIG. 9 is a washer 18 design for an olecranon sled. This washer 18 is similar to the others in many ways, but captures the wire 10 at its extreme end rather than on either side. In addition, this washer 18 has a secondary post 30 that can be impacted into the bone for additional stability.

In FIG. 11, a metal plate 32 has bendable tabs 34 that can be bent to compress and hold a bone fragment 36. Because this complex shape is manufactured from a single bar of metal, it is expensive to manufacture.

As can be observed from these various designs, these washers fix the wire implant to the stable fragment by clamping it against the bone. They require sufficient frictional forces to prevent drift and require a method of implantation in which the wire form implant is applied first, the washer is then applied and both held in place while one or more holes are drilled, measured, tapped and filled with a bone screw. They differ primarily in the mechanism by which they engage and fix the unstable fragment or fragments. Other variations in design of the washer or wire can alter the mechanism of fixation of the unstable fragment but do not alter the basic means by which the various implants are secured to the proximal fragment.

While these implants have different configurations and methods of fixation of the unstable bone fragment, many of these forms share in common a method of securing the implant to the stable bone fragment with a washer and bone screw so as to sandwich the implant between the washer and bone. This method of securing the implant to the bone, however, has several disadvantages.

One disadvantage is that this form of fixation requires additional inventory, since the washer is separate from the implant itself. This adds cost to manufacturing and the additional regulatory requirements of ensuring correct packaging and distribution of the washer.

Another disadvantage is that this method is somewhat cumbersome for the surgical technique. The surgeon must be able to anticipate the position of the hole so that it lines up with the hole in the washer for the bone screw. In addition, the surgeon must simultaneously hold the implant and bone fragments in place, apply and stabilize the washer over the implant and bone fragments, and then insert the bone screw through the washer. Moreover, as the bone screw is inserted the washer has a tendency to rotate which may spin the washer so that it is not optimally oriented for fixation.

Another disadvantage of this method of fixation of the wire or bar to the bone is that the implant is sandwiched and compressed between the washer and the bone in order to prevent the wire or bar from sliding. If the surface of the bone is uneven, there may be inadequate contact of the wire or bar against the bone to allow it to be stabilized. Furthermore, the fixation is dependent on frictional forces between the washer and the implant and the bone and the implant; slight loosening of the screw or inadequate purchase by the screw thread can cause the construct to fail.

In other applications, and as shown, for example, in FIG. 10, a form is described in which the end of the wire or bar is manufactured to expand into the form of a plate with a hole for placement of a screw. In this form, the problems of increased inventory, added complexity to packaging, distribution and regulatory requirements is obviated. In addition, this form is much simpler in terms of surgical technique and is stronger mechanically since the implant is secured directly to the bone and does not depend on frictional constraints. However, this form of production is expensive to make since a complex shape has to be manufactured from a single metal form.

Accordingly, it is an objection of the present invention to address the shortcomings of the prior art devices and methods described herein through the development of a single piece bone fixation device that is economical to manufacture, package and supply, that facilitates ease of use and adjustment by the surgeon using the device, and that meets regulatory and safety concerns.

### SUMMARY OF THE INVENTION

The object of the current invention is to create a composite implant which is formed by a fixation member that is totally or partially fabricated with a polymer and bonded during manufacture to one or more wire elements, comprising wire, bars or tabs that extend from the fixation member and provide bone stabilization. As a result, fewer implant components are needed for the procedure resulting in reduction in packaging, inventory, regulatory requirements, and distribution. In addition, the surgical technique is simplified and direct fixation of the implant to the bone is possible adding strength since the fixation is not dependent on frictional constraints. This structure creates a complete bond between the metal and the polymer reducing concerns with sterilization issues. Finally, the method of manufacture is cheaper than other alternatives that require manufacture of a complex part out of a single block of material, or joining of two parts with crimping or welding.

### BRIEF DESCRIPTION OF THE FIGURES

FIG. 1 shows oblique and top views of a prior art device;

FIG. 2 shows oblique and top views of a prior art device;

FIG. 3 shows side and top views of a prior art device;

FIG. 4 shows top, side and end views of a prior art device;

FIG. 5 shows oblique views of a prior art device;

FIG. 6 shows an oblique view of a prior art device;

FIG. 7 shows oblique and end views of a prior art device;

FIG. 8 shows top and bottom views of a prior art device;

FIG. 9 shows an oblique view of a prior art device;

FIG. 10 shows an oblique view of a prior art device;

FIG. 11 shows a side and top view of a prior art device;

FIG. 12 shows a partial side elevation view of a first preferred embodiment of the present invention, prior to placement of the bone screws;

FIG. 13 shows a partial side elevation view of the first preferred embodiment of the present invention, prior to placement of the bone screws;

FIG. 14 shows a partial top plan view of the first preferred embodiment of the present invention, prior to placement of the bone screws;

FIG. 15 shows a partial side elevation view of the first preferred embodiment of the present invention, subsequent to placement of the bone screws;

FIG. 16 shows a partial side elevation view of the first preferred embodiment of the present invention, subsequent to placement of the bone screws;

FIG. 17 shows a partial top plan view of the first preferred embodiment of the present invention, subsequent to placement of the bone screws;

FIG. 18 shows a partial top plan view of a second preferred embodiment of the present invention, prior to placement of the bone screws;

FIG. 19 shows a partial top plan view of a second preferred embodiment of the present invention, subsequent to placement of the bone screws;

FIG. 20 shows a partial top plan view of a third preferred embodiment of the present invention;

FIGS. 21 and 22 show partial side elevation views of the third preferred embodiment of the present invention;

FIG. 23 shows a partial top plan view of the third preferred embodiment of the present invention;

FIG. 24 shows a partial top plan view of a fourth preferred embodiment of the present invention;

FIG. 25 shows a partial top plan view of a fifth preferred embodiment of the present invention;

FIG. 26 shows a partial top plan view of a sixth preferred embodiment of the present invention;

FIG. 27 shows a partial top plan view of a seventh preferred embodiment of the present invention;

FIG. 28 shows a partial side elevation view of the seventh preferred embodiment of the present invention;

FIGS. 29-31 show side elevation view of additional preferred embodiments of the present invention;

FIG. 32 shows a partial top plan view of an additional preferred embodiment of the present invention; and

FIGS. 33-34 show partial side elevation views of the preferred embodiment of FIG. 32.

### DETAILED DESCRIPTION OF THE FIGURES

In the figures, the lighter shaded regions indicate structure and/or components which are hidden from view behind other structure and/or components.

The present inventive fixation device 100 generally consists of a wire element 102 used to penetrate or buttress stable and/or unstable bone fragments on opposite sides of a fracture site or portion of a first and a second bone, and a fixation member 108 having at least a first portion composed of a polymeric material, such as a poly ether ether ketone (PEEK), that is permanently and securely bonded to the wire element 102. The fixation member 108 comprises at least a first aperture for receiving a fastener, such as a bone screw, for affixation of the fixation member 108 to at least a stable bone fragment or a first bone. The preferred embodiments disclosed herein describe and show different configurations of apertures for receiving the fasteners, different configurations of the fixation member 108, and different configurations of the wire element 102.

In all of the preferred embodiments described below, fewer implant components are needed for bone fixation procedures resulting in reduction in packaging, inventory, regulatory requirements, and distribution. In addition, the surgical technique is simplified and direct fixation of the implant to the bone is possible adding strength since the fixation is not dependent on factional constraints. This structure creates a complete bond between the wire element and the polymer, reducing concerns with sterilization issues. Finally, the method of manufacture is less expensive than other alternatives that require manufacture of a complex part out of a single block of material, or joining of two parts with crimping or welding.

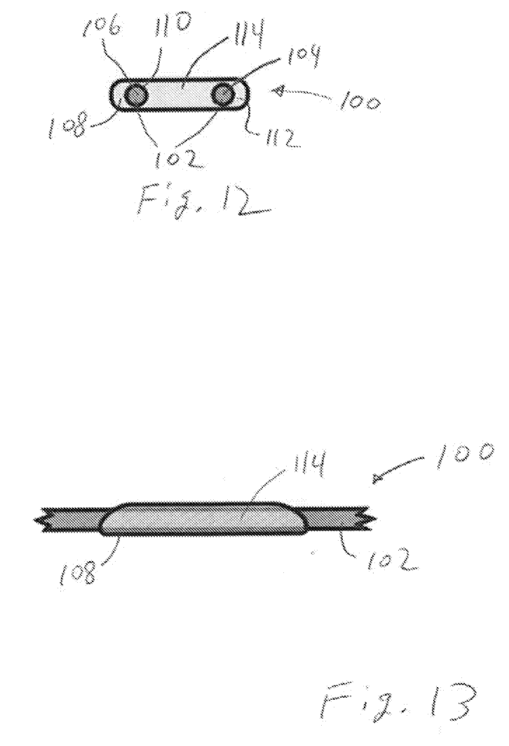

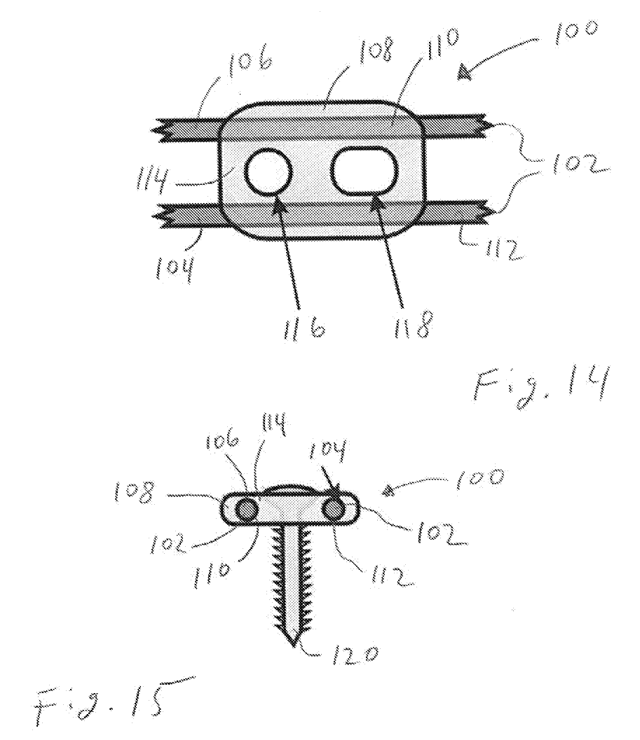

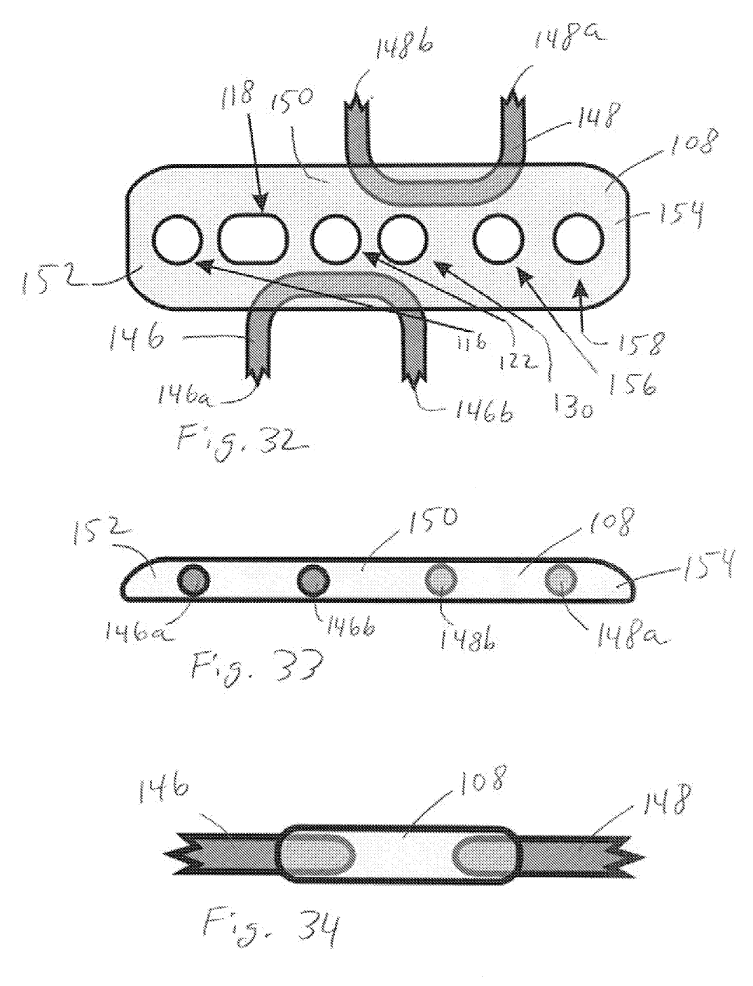

FIGS. 12-34 show preferred embodiments of the present inventive bone fixation device 100. Like reference numerals designate corresponding structures throughout the different views and preferred embodiments. Referring now to FIGS. 12-17, a first preferred embodiment of the present inventive fixation device 100 is shown. The fixation device comprises a continuous, wire element 102 formed with a first and a second longitudinally extending leg 104, 106. The term “wire” or “wire element” as an art recognized term and covers elements having circular or rectangular cross-sections and commonly referred to as pins, wires or bars. A polymerized fixation member 108 is formed and bonded to one or more of the legs 104, 106. In this embodiment, the fixation member 108 takes the form of a substantially rectangular plate. The legs 104, 106 are received by a first and a second channel 110, 112 bored into or integrally formed with the body 114 of the fixation member 108.

Several implantable medical grade polymers exist and could be used in the fabrication of the fixation member 108. These polymers are all capable of facilitating the physical bonding of the fixation member 108 to one or more of the legs 104, 106, thereby eliminating the need for a separate, free fixation member. One such polymer is polyether ether ketone (PEEK). PEEK exhibits physical and chemical properties that enable it to be heated and molded into a predefined shape and then allowed to cool around a metal part to create a composite structure. While PEEK is disclosed for use in this first preferred embodiment, it is contemplated that other medical grade polymers exhibiting similar chemical and physical properties as PEEK can be used while achieving the desired result.

Binding the PEEK fixation member 108 to the legs 104, 106 can be done in multiple ways and still achieve the same final product, namely a uniform, composite structure. In a first method, the PEEK is heated until it is liquefied or semi-fluid or molten. The wire element 102 is then placed into the form holding the heated PEEK raw material and then the PEEK is allowed to cool and harden. In a second method, the body 114 of the fixation member 108 is prefabricated from PEEK and the first channel 110 or the first and the second channels 110, 112 are formed or bored into the body 114 for receiving the legs 104, 106. The fixation member 108 is then heated to expand its size, and consequently the size of the first and the second channels 110, 112. The legs 104, 106 may also be cooled to temporarily decrease their diameter. The legs 104, 106 are passed through the channels 110, 112 in the body 114. Both the fixation member 108 and the legs 104, 106 are allowed to return to room temperature, enabling the diameter of the legs 104, 106 to expand and the washer 108, and particular the diameter of the channels 110, 112, to contract, resulting in a solid bond between the wire element 102 and the fixation member 108.

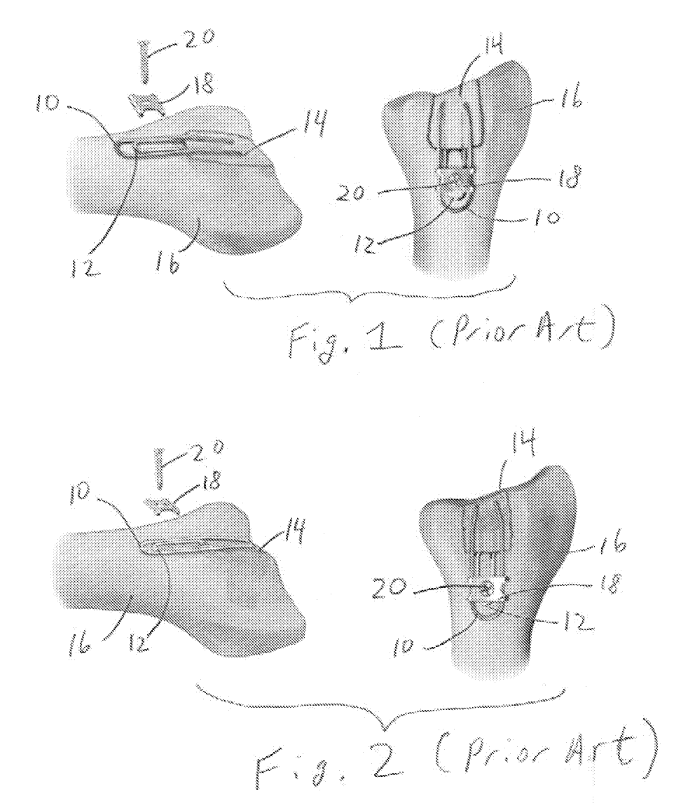

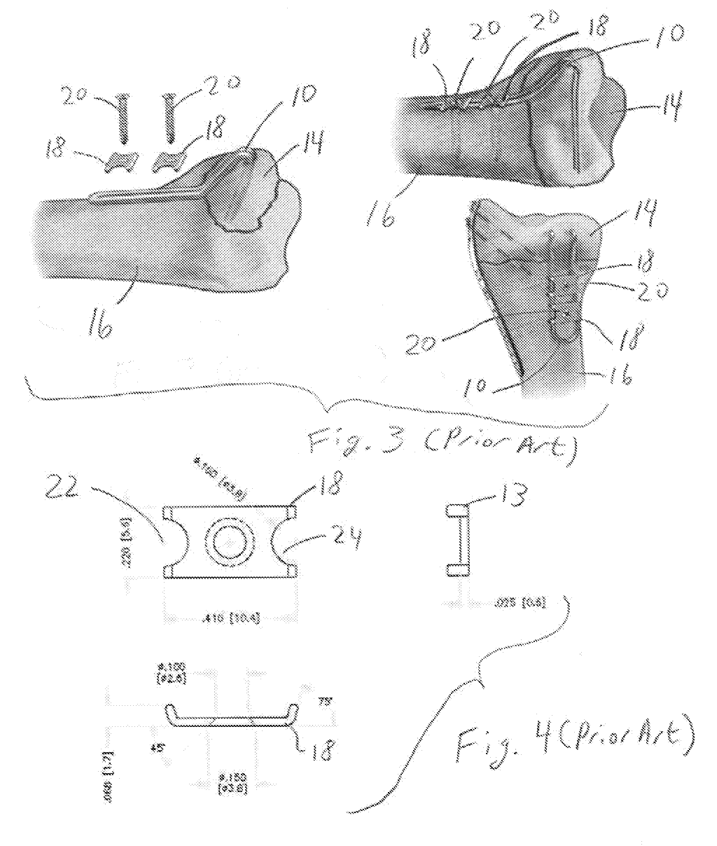

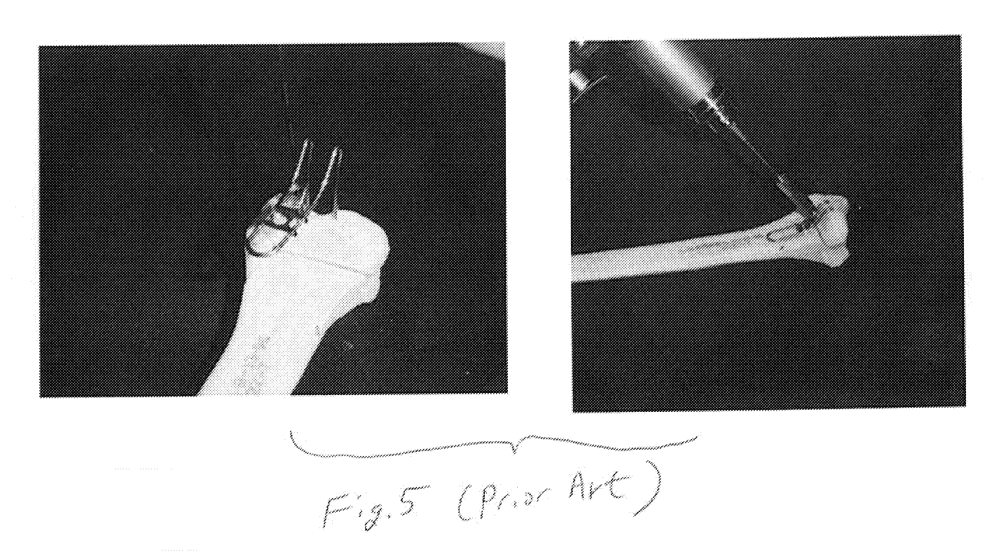

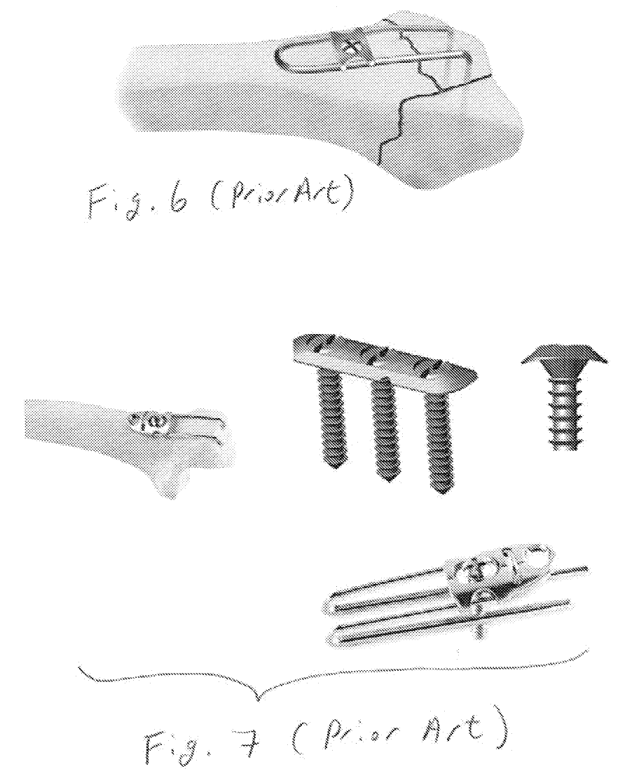

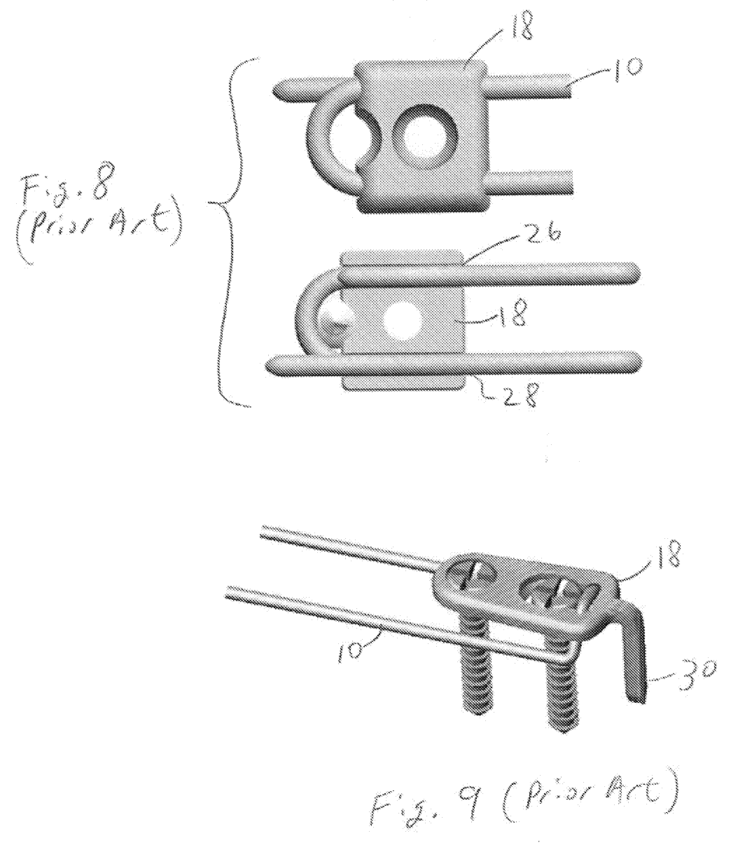

In the present embodiment, the first and a second aperture 116, 118 are bored into the body 114 or are integrally formed with the fixation member 108. The first and second apertures 116, 118 are configured to receive a fastener for securing the fixation device 100 to the bone. In the present embodiment, the first aperture 116 is a substantially circular bore and the second aperture 118 is elliptical or an elongated circle that enables the fixation device 100 to be slid along its length to adjust the position of the device 100 with respect the bone fragment onto which the fixation member 108 is affixed. The first and second apertures 116, 118 are configured to receive a fastener such as a locking or non-locking bone screw 120a, 120b. As is known in the art, locking bone screws may typically employ any of a variety of locking mechanisms, such as a threaded central bore proximate the screw head. When a cooperating locking member is threadedly advanced into the central bore, the larger cross-section of the bone screw's head region causes the head region to grip the surrounding aperture through which the screw has been inserted with a factional fit. Other locking mechanisms, such as, for example, cooperating threads on the screw heads and receiving bores, may alternatively be employed. The configuration and arrangement of the apertures 116, 118 could, of course, be different without changing the spirit or scope of the invention. Moreover, the legs 104, 106 are shown extending from the body 114 without defining the shape or contour of the wire element 102. It is understood that the legs 104, 106 can assume any of the shapes or functions shown in FIGS. 1-9 or in FIG. 29-31.

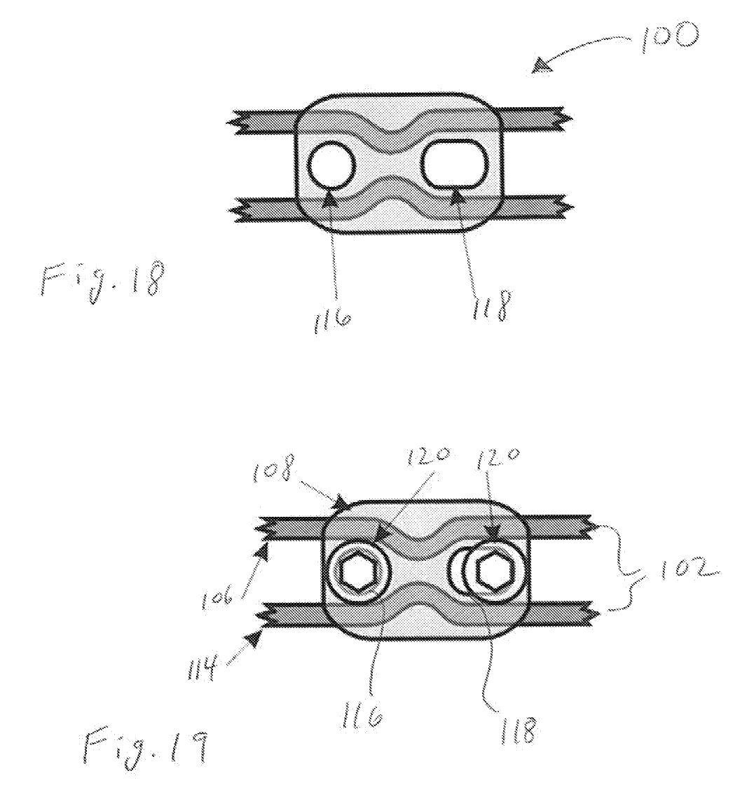

Referring to FIGS. 18 and 19, a second preferred embodiment of the present inventive fixation device 100 is shown. In this embodiment, the first and the second legs 104, 106 of the wire element 102 are non-linear. This non-linear configuration provides improved rotational stabilization of the device 100 when the device 100 is in use and secured to a bone or bone fragment. Because of the non-linear configuration of the first and the second legs 104, 106, the device 100 is formed by heating the polymer raw material used to form the fixation member 108 until it liquefies or becomes semi-liquid or molten, then placing the wire element 102 into position within the heated polymer material, and then allowing the polymer material to harden.

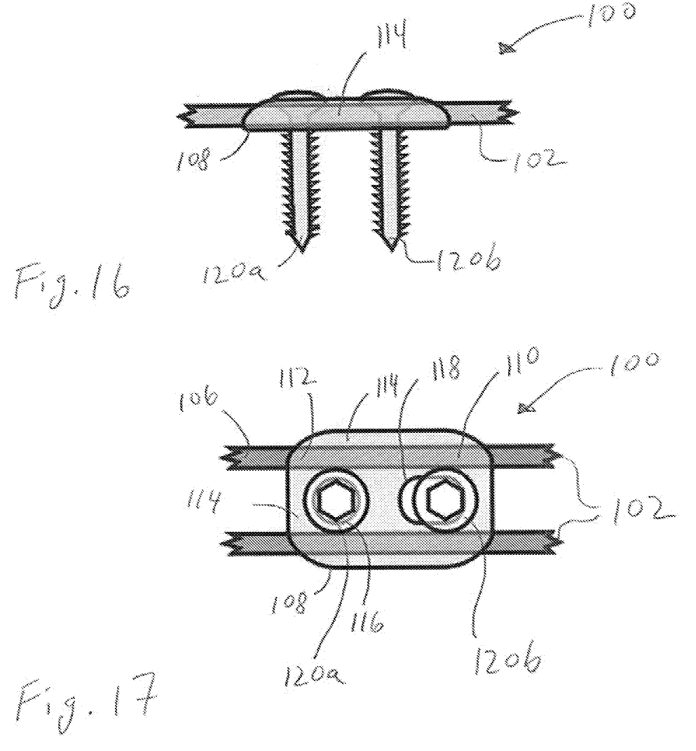

The fixation device 100 of this embodiment also includes a first and a second aperture 116, 118 for receiving a fastener 120, such as a locking or non-locking bone screw. Similar to the embodiment shown in FIGS. 14 and 17, the fixation member 108 is shown to comprise a plate-like structure having a substantially rectangular shape to accommodate the first and second apertures 116, 118. The sizing and configuration of the first and second apertures 116, 118 is the same as those described and shown in FIGS. 14 and 17. Although, in this embodiment, a fixation member with two apertures is illustrated, fixation members with one aperture, as well as those with more than two apertures, are likewise contemplated by the present invention.

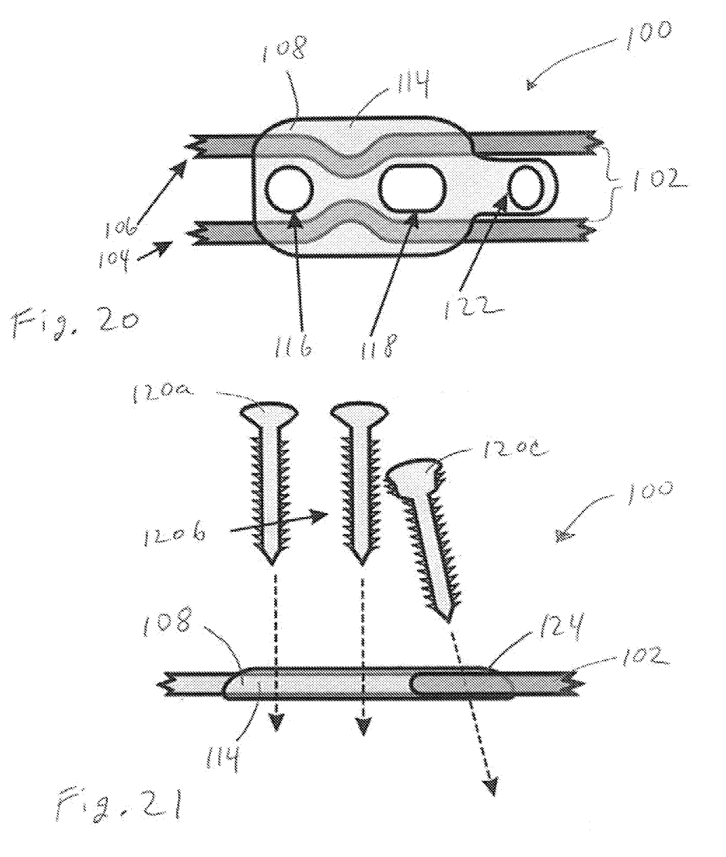

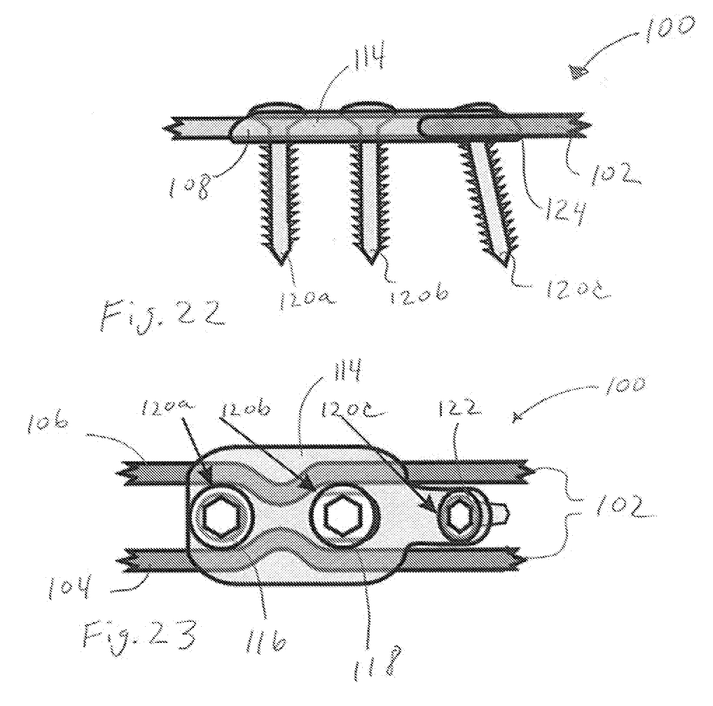

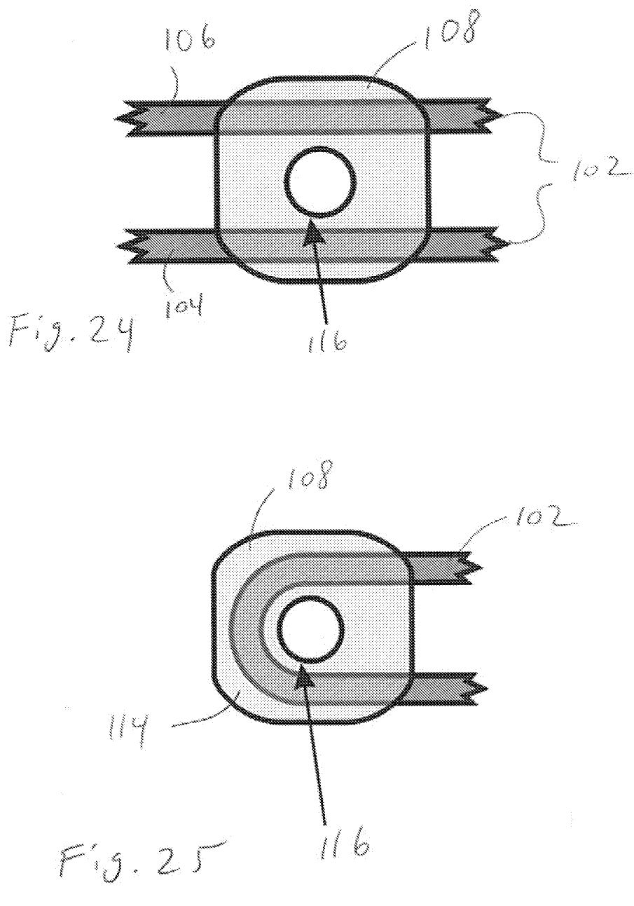

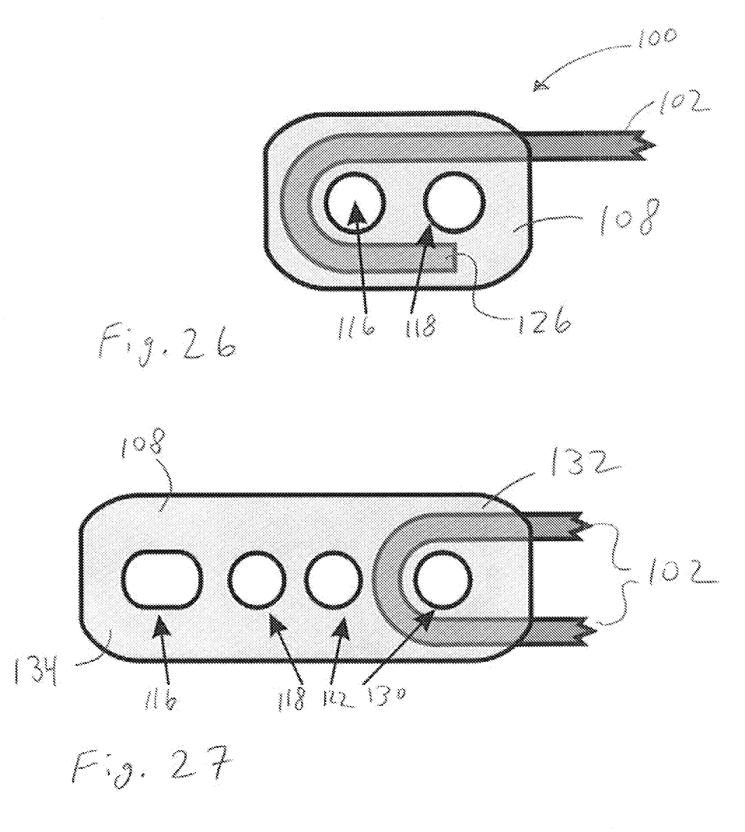

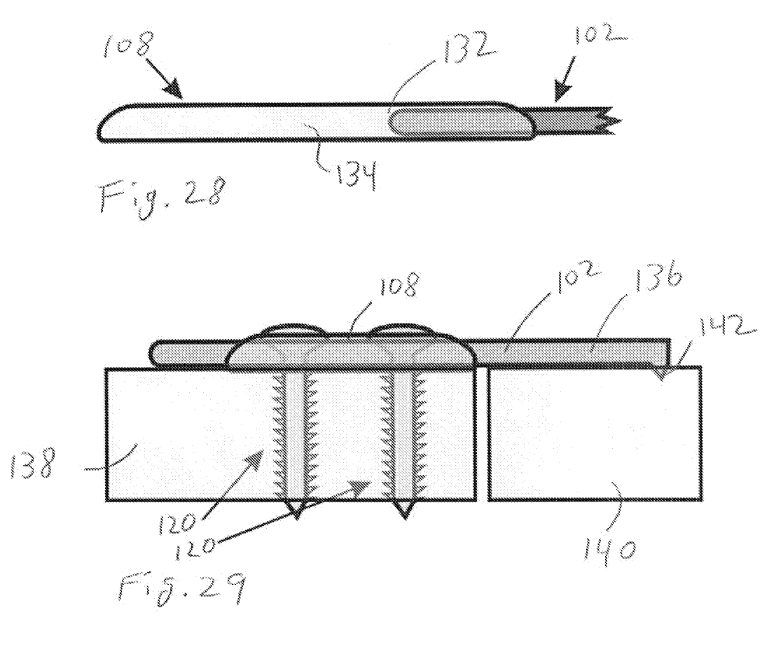

FIGS. 20-23 show a third preferred embodiment of the present inventive fixation device 100. In this embodiment, the fixation member 108 comprises a first 116, a second, 118, and a third 122 aperture for receiving a fastener 120a, 120b, 120c, such as a locking or non-locking bone screw. The apertures 116, 118, 122 may take the form of slots or holes. In this embodiment, the first and third apertures 116, 122 are simple circular bores and the second aperture 118 is a slot. Utilization of the third aperture 122 enables the fixation member 108 to span the fracture site and enable to fasteners to purchase the stable and unstable fragments of the fractured bone. This is accomplished by integrally forming an extension region 124 with the body 114 of the fixation member 108 (FIGS. 20, 22) or by enlarging the body 114 to accommodate the third aperture 122.

As is shown in FIG. 21, the locking bone screw 120c has a threaded head portion 126 as well as a threaded shank portion 128. Use of the polymer material, such as PEEK, to form the fixation member 108, enables the threaded head portion 124 of the fastener 120c to securely engage the sidewalls of the third aperture 122. In addition, while this third preferred embodiment of the present inventive fixation device 100 is shown as comprising a wire element 102 having a non-linear first and second leg 104, 106, it is also contemplated that the first and second leg 104, 106 may also be linear.

FIGS. 24 and 25 show other preferred embodiments of the present inventive fixation device 100 wherein the fixation member 108 comprises only a first aperture 116 for receiving a fastener. In the embodiment of FIG. 25, the wire element 102 does not extend completely through the fixation member 108, but instead loops within the body 114 of the fixation member 108 and around the first aperture 116. The fixation member 108 in both of these embodiments is washer-like in configuration, requiring less raw material to form a body 114 with substantial surface area and volume to accommodate the first aperture 116.

In FIG. 26, a wire element comprising a single leg 104 is formed and bonded to the fixation member 108. The fixation member 108 comprises a first and a second aperture 116, 118 for receiving a fastener (not shown) such as a locking or non-locking bone screw. Similar to the embodiment of FIG. 25, the wire element 102 does not extend completely through the fixation member 108, but instead loops around the first aperture 116. The wire element 102 then terminates at an end 126 in the fixation member 108. Like the fixation devices 100 described and shown in FIGS. 12-25, the form of the exposed end of the wire element 102 is not shown, but could take any shape or form. For example, this embodiment would have particular utility in combination with the fixation device shown in FIG. 10.

FIGS. 27 and 28 show yet another embodiment of the present inventive fixation device 100. The device comprises a fixation member 108 comprising a first 116, a second 118, a third 122, and a fourth 130 aperture for receiving a fastener, such as a locking or non-locking bone screw. Similar to the previous embodiments, the apertures 116, 118, 122, 130 can take the form of a slot or a bore. In the present embodiment, the first aperture 116 comprises a slot and the second 118, third 122, and fourth 130 apertures comprise substantially circular bores. It is contemplated that the fixation member 108 is composed solely from a polymer material, such as PEEK, or can be combination of a first portion 132 composed from a polymer material, such as PEEK, and a second portion 134 from a metal, such as stainless steel or titanium alloy. When the member comprises a first and second portion 132, 134, the member is formed by first heating the polymer material to a liquefied or semi-liquid or molten state, positioning the metal, second portion 134 and wire element 102, and then allowing the polymer material to cool and harden, securing the metal second portion 134 and wire element 102 in place.

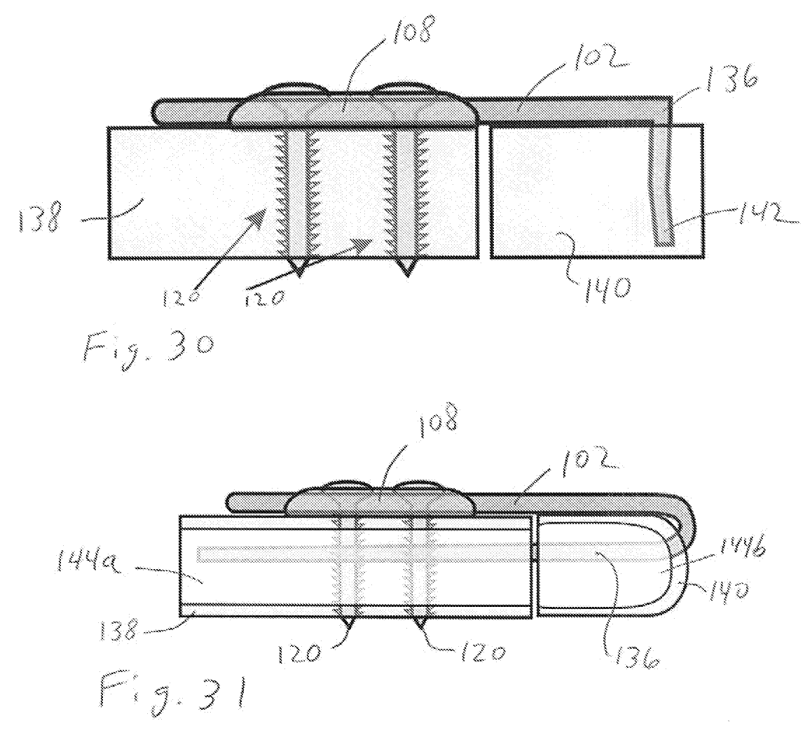

FIGS. 12-28 show the polymer fixation member 108 and a portion of the wire element 102, but do not show possible forms that the wire element 102 can take. FIGS. 29-31 show some possible applications that the wire element 102 can take with any of the previously described forms of the fixation member 108. In FIG. 29, the end 136 of the wire element 102 extends over the surface of a first bone fragment 138 to a second bone fragment 140 and terminates into a tip 142 that provides a surface buttress on the second bone fragment 140. In FIG. 30 the wire element 102 terminates at an end 136 that penetrates the second bone fragment 140 to achieve direct stabilization of the bone 140. In FIG. 31 the end 136 of the wire element 102 penetrates the second bone fragment 140 and then courses within the intramedullary canal 144a, 144b between the second bone fragment 140 and the first bone fragment 138. In the three embodiments described herein, the fixation member 108 includes a first and a second aperture 116, 118 for receiving a fastener 120. In this configuration, the fixation member 108 is secured to only the first bone fragment 138. However, it is also contemplated that the surface area and volume of the fixation member 108 can be expanded to accommodate additional apertures. For a person skilled in the art, other possible applications of the end of the wire or bars are possible without limiting the spirit or scope of the invention.

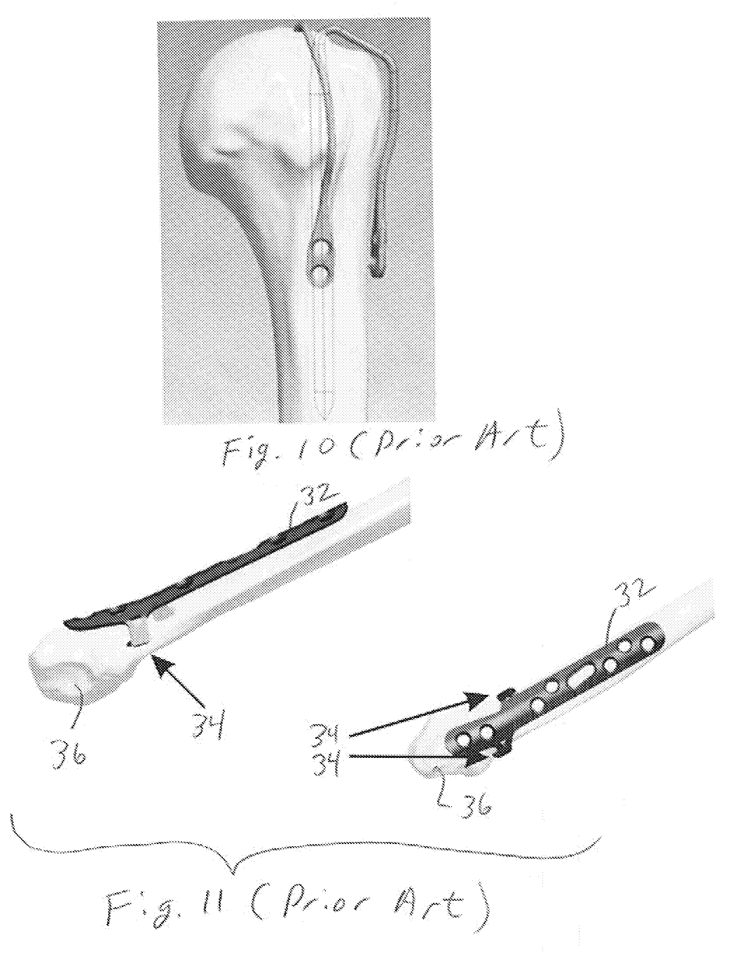

FIGS. 32-34 show yet another preferred embodiment of the present invention, addressing at least the shortcomings of prior art devices similar to that shown in FIG. 11. In this embodiment, the fixation member 108 incorporates at least a first and second wire element 146, 148 extending from opposite sides of the fixation member 108. The fixation member 108 can be constructed entirely out of a polymer, such as PEEK, or can comprise a combination of a polymer section 150 in the area that binds the wire elements 146, 148 and metal sections 152, 154 on opposite sides of the polymer section 150. In this example, the wire elements 146, 148 are shown extending from either side of the fixation member 108, however, it is contemplated that only a single wire element 146 or 148 extending from only one side of the fixation member 108 can be used. The fixation device 100 of this embodiment is advantageous over the prior art, such as the device shown in FIG. 11, because the fixation member 108 does not require complex manufacturing of the tabs 34 shown in FIG. 11.

It is to be understood that even though numerous characteristics and advantages of the present inventive fixation device have been set forth herein, together with the details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

I claim:

1) A bone fixation device for affixing to a first and second bone segment the fixation device comprising:
a fixation member having at least a first aperture for receiving a fastener to couple the fixation member to the first bone segment; and
a first wire element integrally bonded to the fixation member for engaging the second bone segment.

2) The bone fixation device of claim 1 wherein at least one of the fixation member and the first wire element comprises a nonmetallic material.

3) The bone fixation device of claim 2 wherein the fixation member comprises at least a first portion comprised of a polymer material.

4) The bone fixation device of claim 3 wherein the polymer material comprises polyether ether ketone.

5) The bone fixation device of claim 3 wherein the fixation member further comprises a second portion comprised of a metal.

6) The bone fixation device of claim 1 wherein a first length of the first wire element is encased within the fixation member and a second length of the of the first wire element extends from the fixation member to engage the first or the second bone segment.

7) The bone fixation device of claim 1 wherein the first aperture overlies the first bone segment and a second aperture is operably positioned so as to enable fixation of the second bone segment

8) The bone fixation device of claim 7 wherein the first bone segment comprises a stable bone segment and the second bone segment comprises an unstable bone fragment on the opposite side of a fracture site from the stable bone portion.

9) The bone fixation device of claim 1 wherein the fastener is a bone screw.

10) The bone fixation device of claim 9 wherein the fastener is a locking bone screw.

11) The bone fixation device of claim 9 wherein the fastener is a non-locking bone screw.

12) The bone fixation device of claim 1 wherein the first wire element comprises a rod.

13) The bone fixation device of claim 1 wherein the first wire element comprises a wire.

14) The bone fixation device of claim 1 wherein the first wire element comprises a bar.

15) The bone fixation device of claim 1 wherein the first wire element comprises a tab.

16) The bone fixation device of claim 1 wherein the first wire element is bendable or malleable.

17) The bone fixation device of claim 1 wherein a first length of the first wire element is linear.

18) The bone fixation device of claim 1 wherein a first length of the first wire element is non-linear.

19) The bone fixation device of claim 1 wherein the fixation member comprises a fixation plate.

20) The bone fixation device of claim 1 wherein the fixation member comprises a washer.

21) The bone fixation device of claim 1 wherein the wire element buttresses the first or second bone segment.

22) The bone fixation device of claim 1 wherein the wire element penetrates at least one of the first and second bone segments.

23) The bone fixation device of claim 1 further comprising a second wire element.

24) The bone fixation device of claim 23 wherein the first and the second wire elements are disposed, on opposite sides of the fixation member.

25) A bone fixation device for affixing to a first and second bone segment, the bone fixation device comprising:
a fixation member comprising at least a first portion composed from a polymer material; and
a first wire element comprising a first length encased by the first portion of the fixation member and a second length extending from the fixation member to securely engage the first or the second bone segment.

26) The fixation device of claim 25 wherein the fixation member further comprises a first aperture for receiving a fastener to couple the fixation member to the first bone segment.

27) The bone fixation device of claim 26 further comprising a second aperture operably positioned so as to enable fixation of the second bone segment.

28) The fixation device of claim 25 wherein the first portion of the fixation member is composed of a polyether ether ketone.

29) The fixation device of claim 25 wherein the fixation member further comprises a second portion composed of metal.

30) The bone fixation device of claim 25 wherein the first wire element comprises a rod.

31) The bone fixation device of claim 25 wherein the first wire element comprises a wire.

32) The bone fixation device of claim 25 wherein the first wire element comprises a bar.

33) The bone fixation device of claim 25 wherein the first wire element comprises a tab.

34) The bone fixation device of claim 25 wherein the first wire element is bendable or malleable.

36) The bone fixation device of claim 25 wherein the first length of the first wire element is linear.

36) The bone fixation device of claim 25 wherein the first length of the first wire element is non-linear.

37) The bone fixation device of claim 25 wherein the first wire element buttresses at least one of the first and second bone segments.

38) The bone fixation device of claim 25 wherein the first wire element penetrates the first or second bone segment.

39) The bone fixation device of claim 25 further comprising a second wire element.

40) The bone fixation device of claim 39 wherein the first and the second wire elements are disposed on opposite sides of the fixation member.

41) The bone fixation device of claim 25 wherein the fastener is a bone screw.

42) The bone fixation device of claim 25 wherein the fastener is a locking bone screw.

43) The bone fixation device of claim 25 wherein the fastener is a non-locking bone screw.

44) The bone fixation device of claim 26 wherein the fixation member comprises a fixation plate.

45) The bone fixation device of claim 25 wherein the fixation member comprises a washer.
